(19) United States
(12) Patent Application Publication
(10) Pub. No.: US 2008/0132798 A1
(43) Pub. Date: Jun. 5, 2008

(54) WIRELESS HEADSETS AND WIRELESS COMMUNICATION NETWORKS FOR HEART RATE MONITORING

(75) Inventors: Di-An Hong, Inverness, IL (US); Mark W. Cholewczynski, Wheaton, IL (US); Janice M. Danvir, Arlington Heights, IL (US); Krishna D. Jonnalagadda, Algonquin, IL (US); Francesca Schuler, Des Plaines, IL (US)

Correspondence Address: PRASS & IRVING LLP, 2661 Riva Road, Bldg. 1000, Suite 1044, ANNAPOLIS, MD, 21401, US

(73) Assignee: Motorola, Inc, Schaumburg, IL (US)

(21) Appl. No.: 11/565,259

(22) Filed: Nov. 30, 2006

(51) Int. Cl. A61B 5/024

(52) U.S. Cl. 600/508

(57) ABSTRACT

A multi-functional wireless headset may include a heart rate sensing assembly configured to detect heart rate data of a wearer of the headset, and a wireless communication unit configured to communicate heart rate data to a gateway device.

### TECHNICAL FIELD

The present invention is directed to wireless headsets and wireless networks. More particularly, the present invention is directed to wireless headsets and wireless communication networks for use in heart rate monitoring.

### BACKGROUND

Some conventional heart rate monitors employ sensors that are worn without a chest strap. For example, some conventional heart rate monitors are incorporated into a band, watch, or other device worn on a user's wrist. Such devices typically require a user to press a key or series of keys when it is desired to check heart rate. Some conventional monitors incorporate a sensor in a band that can be wrapped around a user's finger. The sensor is then connected to a wristband or wristwatch, where the data from the sensor is processed and where heart rate information is displayed.

Another conventional heart rate monitor comprises an earlobe clip sensor. The sensor is wired to a separate device that processes data from the sensor and displays heart rate information. Still another conventional device includes a sensor incorporated in a device configured to be grasped by both hands. The data can be processed by the device, and heart rate information can be displayed by the device as well.

Thus, it may be desirable to provide a reliable, wireless heart rate monitor. It may be desirable to monitor heart rate at a body location that is not otherwise used during monitoring. For example, it may be desirable to avoid attaching sensors to the hands, legs, and chest during physical workouts.

One example of a conventional device monitoring device is described in U.S. Patent Publication No. 2003/0233051. The device incorporates an optical probe in an earpiece for measuring heart rate. The earpiece is wired to a portable device that can also be used to play music, such as a Walkman tape player.

It may be desirable to provide a wireless headset that can be used for various audio functions, including audio function related to heart rate monitoring. It may be desirable to provide a wireless network for communicating heart rate data and providing audible and/or visual feedback to the user.

### SUMMARY OF THE INVENTION

According to various aspects of the disclosure, a multi-functional wireless headset may include a heart rate sensing assembly configured to detect heart rate data of a wearer of the headset, and a wireless communication unit configured to communicate heart rate data to a gateway device.

In some aspects of the disclosure, a wireless headset may comprise an audio communication unit configured to output audio hearable by a wearer of the headset, at least one heart rate sensing assembly configured to detect heart rate data of the wearer, and a wireless communication unit configured to communicate heart rate data to a gateway device.

In accordance with various aspects of the disclosure, a wireless communication network may include a mobile device configured to wirelessly communicate with at least one device and a wireless headset. The headset may comprise an audio communication unit configured to output audio hearable by a wearer of the headset, at least one heart rate sensing assembly configured to detect heart rate data of the wearer, and a wireless communication unit configured to communicate the heart rate data to the mobile device. The network may include at least one auxiliary device configured to wirelessly communicate with the mobile device and/or the headset.

### BRIEF DESCRIPTION OF THE DRAWINGS

FIG. 1 is a diagrammatic view of a single-earpiece headset in accordance with exemplary aspects of the disclosure;

FIG. 2 is a diagrammatic view of a dual-earpiece headset in accordance with exemplary aspects of the disclosure;

FIG. 3 is a diagrammatic view of sensor locations for heart rate detection in accordance with exemplary aspects of the disclosure;

FIG. 4 is a diagrammatic view of a wireless communication network in accordance with exemplary aspects of the disclosure; and

FIG. 5 is a block diagram of a signal processing device for heart rate computing in accordance with exemplary aspects of the disclosure.

### DETAILED DESCRIPTION

An exemplary embodiment of a wireless headset 110 is illustrated in FIG. 1. The wireless headset 110 may be associated with a mobile device and/or a wireless network, as described in more detail below.

The wireless headset 110 may comprise a single earpiece 112 It should be appreciated the single earpiece 112 shown in FIG. 1 is exemplary only and that other forms of single and dual headsets are encompassed by the disclosure.

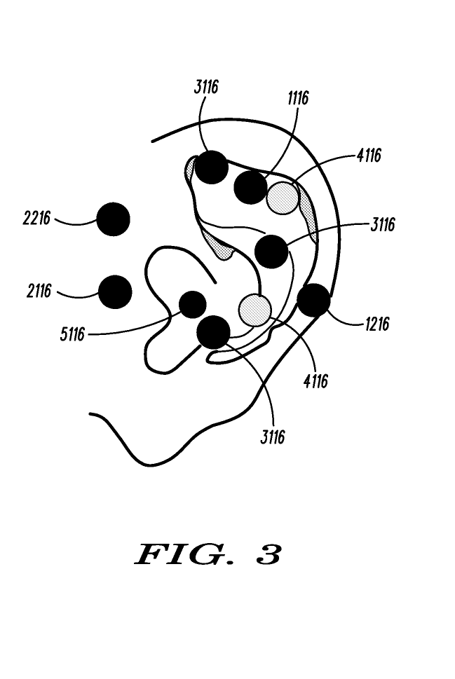

The wireless headset 110 includes a heart rate sensing assembly 114 configured to detect heart rate data of a wearer of the headset 110. The sensing assembly 114 may comprise one or more heart rate sensors 116, such as, for example, photoplethysmograph (PPG) sensors. Positioning of the sensors 116 is discussed below in connection with FIG. 3. The headset 110 may include one or more additional bio-signal sensors (not shown), such as, for example, temperature and/or acoustic sensors, which may enhance the accuracy of heart rate determination.

The headset 110 may include a wireless communication unit 118 configured to communicate heart rate data to a gateway device 130 and/or one or more auxiliary devices 150. The gateway device 130 may comprise any mobile device, such as, for example, a mobile telephone, a personal digital assistant or personal information device, a pager, an mp3 player, an iPod, or the like. The gateway device 130 may be a handheld device or it may be associated with a wristband, armband, or the like. The auxiliary device 150 may comprise a display, such as, for example, one that can be associated with an armband or wristband and worn by the user, or any other type of output device, for example, any device that can provide an audible and/or visual signal to the user.

The gateway device 130 can receive the heart rate data directly from the headset 110 or via another wireless link (not shown). The wireless communication unit 118 may be configured to communicate via one or more wireless protocols, such as, for example, Bluetooth, ZigBee, and the like.

The headset 110 may include an audio communication unit 120.

The audio configuration unit 120 may output audio hearable by a user/wearer of the headset 1 10. For example, the audio unit 120 can selectively output user-selected audio such as, for example, music, sound associated with video, telephone communications, or the like.

In some aspects, the headset 110 may include a processing unit 122. The processing unit 122 may be configured to determine a heart rate of the user/wearer based on the raw data stream from the sensor. In some aspects, the processing unit 122 is in the gateway device 130 or another device (not shown). According to various aspects, a Petri-Net may be used for distributed control of the gateway device monitoring of heart rate data and providing audible and/or visual feedback of heart rate information to the user via the headset 110, the gateway device 130, or an auxiliary device 150.

According to various aspects, the processing unit 122 may be configured to reduce motion artifacts attributable to movement of the wearer.

For example, the headset 110 may include one or more accelerometers (not shown) and/or one or more gyroscopes (not shown) or other inertial motion sensing devices capable of detecting motion of the user/wearer.

In some aspects, the gateway device 130 and/or the auxiliary device 150 can output audible and/or visual notifications, alerts, or warnings associated with the heart rate of the user as determined by the processing unit 122. Similarly, the audio unit 120 can output audible notifications, alerts, or warnings associated with the heart rate of the user as determined by the processing unit 122.

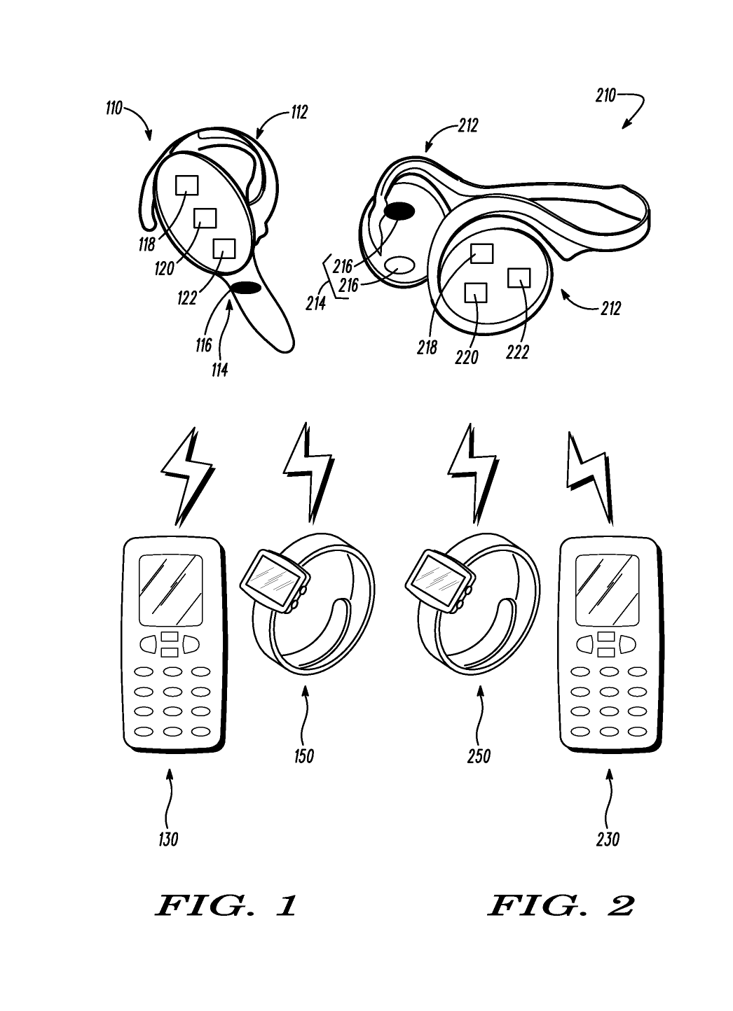

Referring to FIG. 2, an exemplary headset 210 in accordance with various aspects of the disclosure may include dual earpieces 212. It should be appreciated the dual earpiece 212 shown in FIG. 2 is exemplary only and that other forms of single and dual headsets are encompassed by the disclosure.

One or both of the earpieces 212 of the headset 210 may comprise a heart rate sensing assembly 214 configured to detect heart rate data of a wearer of the headset 21 0. Although FIG. 2 illustrates both earpieces 212 having a sensing assembly 214, one of ordinary skill in the art would recognize that the headset 210 could include a sensing assembly 214 in only one of the earpieces 212.

The sensing assembly 214 may comprise one or more heart rate sensors 216, such as, for example, PPG sensors. Positioning of the sensors 216 is discussed below in connection with FIG. 3. The headset 210 may include one or more additional bio-signal sensors (not shown), such as, for example, temperature and/or acoustic sensors, which may enhance the accuracy of heart rate determination.

The headset 210 may include a wireless communication unit 218 configured to communicate heart rate data to a gateway device 230 and/or one or more auxiliary devices 250. The gateway device 230 may comprise any mobile device, such as, for example, a mobile telephone, a personal digital assistant or personal information device, a pager, an mp3 player, an iPod, or the like. The gateway device 230 may be a handheld device or it may be associated with a wristband, armband, or the like. The auxiliary device 250 may comprise a display, such as, for example, one that can be associated with an armband or wristband and worn by the user, or any other type of output device, for example, any device that can provide an audible and/or visual signal to the user.

The gateway device 230 can receive the heart rate data directly from the headset 210 or via another wireless link (not shown). The wireless communication unit 218 may be configured to communicate via one or more wireless protocols, such as, for example, Bluetooth, ZigBee, and the like.

The headset 210 may include an audio communication unit 220.

The audio configuration unit 220 may output audio hearable by a user/wearer of the headset 210. For example, the audio unit 220 can selectively output user-selected audio such as, for example, music, sound associated with video, telephone communications, or the like.

In some aspects, the headset 210 may include a processing unit 222. The processing unit 222 may be configured to determine a heart rate of the user/wearer based on the sensed heart rate data. In some aspects, the processing unit 222 may be in the gateway device 230 or another device (not shown). According to various aspects, a Petri-Net may be used for distributed control of the gateway device monitoring of heart rate data and providing audible and/or visual feedback of heart rate information to the user via the headset 210, the gateway device 230, or an auxiliary device 250.

According to various aspects, the processing unit 222 may be configured to reduce motion artifacts attributable to movement of the wearer.

For example, the headset 210 may include one or more accelerometers (not shown) and/or one or more gyroscopes (not shown) or other inertial motion sensing devices capable of detecting motion of the user/wearer.

In some aspects, the gateway device 230 and/or the auxiliary device 250 can output audible and/or visual notifications, alerts, or warnings associated with the heart rate of the user as determined by the processing unit 222. Similarly, the audio unit 220 can output audible notifications, alerts, or warnings associated with the heart rate of the user as determined by the processing unit 222.

Referring to FIG. 3, the heart rate sensors 116, 216 may be disposed on various areas of the headset 110, 210 such that the heart rate sensors 116, 216 will be positioned substantially at selected locations on or proximate the wearer's ear in an attempt to optimize heart rate detection. According to various aspects, heart rate sensors may be positioned on the earpiece so as to detect heart rate data substantially from a portion 1116, 3116, 1216 of the ear other than the earlobe. In some aspects, one of the sensors may be positioned on the earpiece so as to detect heart rate data substantially from a region 2116, 2216 of the wearer other than the ear. Heart rate data can also be detected substantially at other portions 4116 of the back of the ear. In various aspects, heart rate data can be measured at an ear canal 5116 via direct contact PPG.

For example, as shown in FIG. 3, a stereo headset, for example, headset 210, may include two sensors associated with each earpiece 212 so as to detect heart rate data substantially at a portion 1216 of the ear or a region 2216 away from the ear. The sensors may be positioned on the headset 210 to face the front of the wearer's ear, i.e., the surface of the ear facing away from the wearer's head. It should be appreciated that the headset 210 may include two sensors associated with only one earpiece, one of the sensors associated with each earpiece, or one of the sensors associated with only one earpiece.

According to some aspects of the disclosure, a mono headset, for example, headset 110, may include one or more of sensors positioned on the headset 110 so as to detect heart rate data substantially at a portion 1116 of the ear or a region 2116 away from the ear. The sensors may be positioned on the headset 110 to face the front of the wearer's ear. According to various aspects of the disclosure, a mono headset may include one or more of sensors positioned on the headset 110 to face the back of the wearer's ear and detect heart rate data substantially at a portion 3116 of the back of the ear.

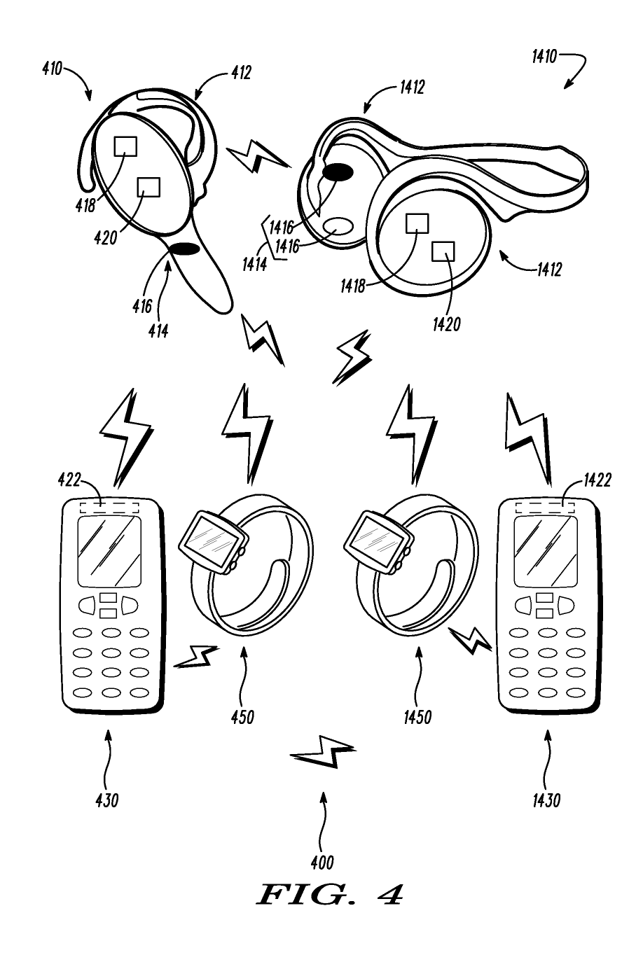

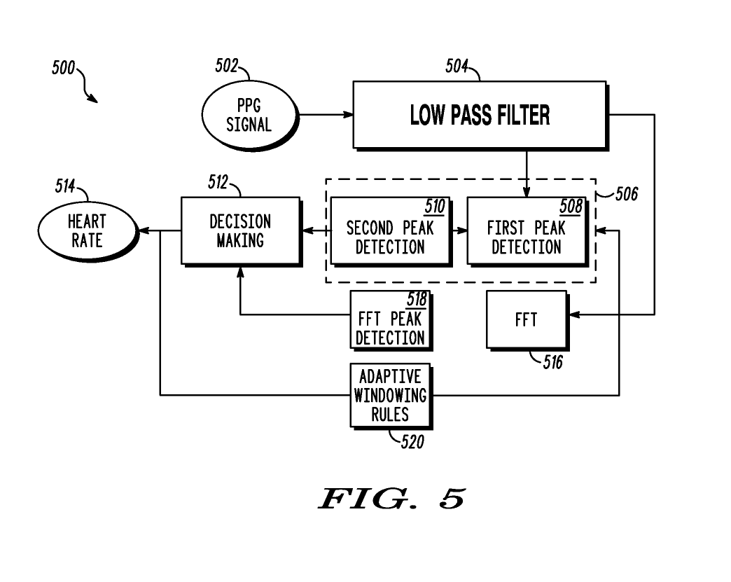

Referring now to FIG. 4, according to various aspects of the disclosure, a wireless communication network 400, for example, a body area network, may include a mobile device 430 and a wireless headset 410. Although the wireless headset 410 is illustrated as a single-earpiece headset similar to the headset 110 described in relation to FIG. 1, it should be appreciated that the headset 410 may comprise a dual-earpiece headset similar to the headset 210 described in relation to FIG. 2. The headset 410 and mobile device 430 are configured to wirelessly communicate with one another.

The wireless headset 410 includes a heart rate sensing assembly 414 configured to detect heart rate data of a wearer of the headset 410. The sensing assembly 414 may comprise one or more heart rate sensors 416, such as, for example, PPG sensors. Positioning of the sensors 416 was discussed above in connection with FIG. 3. The headset 410 may include one or more additional bio-signal sensors (not shown), such as, for example, temperature and/or acoustic sensors, which may enhance the accuracy of heart rate determination.

The headset 410 may include a wireless communication unit 418 configured to wirelessly communicate heart rate data to the mobile device 430 and/or one or more auxiliary devices 450. The mobile device 430 may comprise a handheld device, such as, for example, a cellular telephone, a smartphone, a personal digital assistant or personal information device, a pager, an mp3 player, an iPod, or the like.

The auxiliary device 450 may comprise a display, such as, for example, one that can be associated with an armband or wristband and worn by the user, or any other type of output device, for example, any device that can provide an audible and/or visual signal to the user. The auxiliary device 450 and/or the mobile device 430 may be configured to display information associated with the sensed heart rate data. The auxiliary device 450 may be configured to wirelessly communicate with the mobile device 430 and/or the headset 410 via one or more wireless protocols, such as, for example, Bluetooth, ZigBee, and the like.

The mobile device 430 can receive the heart rate data directly from the headset 410 or via another wireless link (not shown). The wireless communication unit 418 may be configured to communicate with the mobile device 430 and/or the auxiliary device 450 via one or more wireless protocols, such as, for example, Bluetooth, ZigBee, and the like.

The headset 410 may include an audio communication unit 420. The audio configuration unit 420 may output audio hearable by a user/wearer of the headset 410. For example, the audio unit 420 can selectively output user-selected audio such as, for example, music, sound associated with video, telephone communications, or the like.

In some aspects, the mobile device 430 may include a processing unit 422. The processing unit 422 may be configured to determine a heart rate of the user/wearer based on the sensed heart rate data. In some aspects, the processing unit 422 may be in the headset 410. According to various aspects, a Petri-Net may be used for distributed control of the mobile device monitoring of heart rate data and providing audible and/or visual feedback of heart rate information to the user via the headset 410, the mobile device 430, or the auxiliary device 450.

According to various aspects, the processing unit 422 may be configured to reduce motion artifacts attributable to movement of the wearer.

For example, the headset 410 may include one or more accelerometers (not shown) and/or one or more gyroscopes (not shown) or other inertial motion sensing devices capable of detecting motion of the user/wearer.

In some aspects, the mobile device 430 and/or the auxiliary device 450 can output audible and/or visual notifications, alerts, or warnings associated with the heart rate of the user as determined by the processing unit 422. Similarly, the headset 410 can output audible notifications, alerts, or warnings associated with the heart rate of the user as determined by the processing unit 422.

The mobile device 430 may include a recordable medium 432, such as for example, ROM, RAM, or any storage device. The recordable medium 432 may include an application for heart rate monitoring. It should be appreciated that, alternatively or additionally, a recordable medium 432 may be disposed on the headset 410 and/or the auxiliary device 450. The application may be commenced at the mobile device by a user via voice, data, text, or phrase selection.

FIG. 4 also illustrates a second wireless communication network 1400, for example, a body area network, may include a second mobile device 1430 and a second wireless headset 1410. Although the wireless headset 1410 is illustrated as a dual-earpiece headset similar to the headset 210 described in relation to FIG. 2, it should be appreciated that the headset 1410 may comprise a single-earpiece headset similar to the headset 110 described in relation to FIG. 1. The headset 1410 and mobile device 1430 are configured to wirelessly communicate with one another.

The wireless headset 1410 may include a heart rate sensing assembly 1414 configured to detect heart rate data of a wearer of the headset 1410. The sensing assembly 1414 may comprise one or more heart rate sensors 1416, such as, for example, PPG sensors. Positioning of the sensors 1416 was discussed above in connection with FIG. 3. The headset 1410 may include one or more additional bio-signal sensors (not shown), such as, for example, temperature and/or acoustic sensors, which may enhance the accuracy of heart rate determination.

The headset 1410 may include a wireless communication unit 1418 configured to wirelessly communicate heart rate data to the mobile device 1430 and/or one or more auxiliary devices 1450. The mobile device 1430 may comprise a handheld device, such as, for example, a cellular telephone, a smartphone, a personal digital assistant or personal information device, a pager, an mp3 player, an iPod, or the like.

The auxiliary device 1450 may comprise a display, such as, for example, one that can be associated with an armband or wristband and worn by the user, or any other type of output device, for example, any device that can provide an audible and/or visual signal to the user. The auxiliary device 1450 and/or the mobile device 1430 may be configured to display information associated with the sensed heart rate data. The auxiliary device 1450 may be configured to wirelessly communicate with the mobile device 1430 and/or the headset 1410 via one or more wireless protocols, such as, for example, Bluetooth, ZigBee, and the like.

The mobile device 1430 can receive the heart rate data directly from the headset 1410 or via another wireless link (not shown). The wireless communication unit 1418 may be configured to communicate with the mobile device 1430 and/or the auxiliary device 1450 via one or more wireless protocols, such as, for example, Bluetooth, ZigBee, and the like.

The headset 1410 may include an audio communication unit 1420. The audio configuration unit 1420 may output audio hearable by a user/wearer of the headset 1410. For example, the audio unit 1420 can selectively output user-selected audio such as, for example, music, sound associated with video, telephone communications, or the like.

In some aspects, the mobile device 1430 may include a processing unit 1422. The processing unit 1422 may be configured to determine a heart rate of the user/wearer based on the sensed heart rate data. In some aspects, the processing unit 1422 is in the headset 1410. According to various aspects, the processing unit 1422 may be configured to reduce motion artifacts attributable to movement of the wearer. For example, the headset 1410 may include one or more accelerometers (not shown) and/or one or more gyroscopes (not shown) or other inertial motion sensing devices capable of detecting motion of the user/wearer.

In some aspects, the mobile device 1430 and/or the auxiliary device 450 can output audible and/or visual notifications, alerts, or warnings associated with the heart rate of the user as determined by the processing unit 1422. Similarly, the headset 1410 can output audible notifications, alerts, or warnings associated with the heart rate of the user as determined by the processing unit 1422.

The mobile device 1430 may include a recordable medium 1432, such as for example, ROM, RAM, or any storage device. The recordable medium 1432 may include an application for heart rate monitoring. The application may be commenced at the mobile device by a user via voice, data, text, or phrase selection.

The mobile device 1430 may be configured to wirelessly communicate with the mobile device 430 and/or the headset 410 via one or more wireless protocols, such as, for example, Bluetooth, ZigBee, and the like. Similarly, mobile device 430 may be configured to wirelessly communicate with the headset 1410 via one or more wireless protocols, such as, for example, Bluetooth, ZigBee, and the like. According to some aspects, the headsets 410, 1410 may be configured to wirelessly communicate with one another via one or more wireless protocols, such as, for example, Bluetooth, ZigBee, and the like. According to various aspects, the headset 1410 and/or the mobile device 1430 may be configured to wirelessly communicate with the auxiliary device 450 via one or more wireless protocols, such as, for example, Bluetooth, ZigBee, and the like. It should be appreciated that the network 400 and second network 1400 can combine to form a wider network, thereby allowing multiple users to share data with each other, with a medical practitioner, and/or with any third party.

Referring now to FIG. 5, a block diagram 500 illustrates a signal processing device for determining heart rate. A PPG signal 502 is processed by a low pass filter 504. The resultant signal is then sent to two processing devices. A first processing device comprises a detection algorithm 506 examining a first peak detection 508 and a second peak detection 5 10. The output of the detection algorithm is then processed by a decision making algorithm 512 where a heart rate determination is made and subsequently output 514.

The second processing device encountered by the output of the low pass filter 504 is a Fast Fourier Transform 516 followed by a Fast Fourier Transform peak detection 518. The output of the Fast Fourier Transform peak detection 518 is processed by the decision making algorithm 512 where the heart rate determination is made and then output 514.

In all cases, the heart rate determination output from the decision making algorithm 512 is also fed back to the detection algorithm 506, but is first processed by an adaptive windowing process 520. The adaptive windowing process 520 dynamically alters the data window size depending on previous heart rate measurements and determinations and based on data quality.

It will be apparent to those skilled in the art that various modifications and variations can be made in the devices and methods of the present disclosure without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A multi-functional wireless headset, comprising:
a heart rate sensing assembly configured to detect heart rate data of a wearer of the headset; and
a wireless communication unit configured to communicate heart rate data to a gateway device.

2. The headset of claim 1, wherein the wireless communication unit is configured to communicate via at least one wireless protocol.

3. The headset of claim 1, where the heart rate sensing assembly comprises a photoplethysmograph sensing assembly.

4. The headset of claim 1, further comprising at least one earpiece configured to be worn on an ear, the heart rate sensing assembly comprising at least one sensor on the earpiece.

5. The headset of claim 4, wherein the at least one sensor is positioned on the earpiece so as to detect heart rate data from at least one of a portion of the ear other than the earlobe, an ear canal, and a region of the wearer other than the ear.

6. The headset of claim 1, further comprising at least one inertial motion sensing device capable of detecting motion of the wearer, the detected motion being usable to reduce motion artifacts attributable to movement of the wearer from the detected heart rate data.

7. The headset of claim 1, further comprising a processing unit configured to receive the detected heart rate data and determine a heart rate of wearer based on the heart rate data.

8. A wireless headset, comprising:
an audio communication unit configured to output audio hearable by a wearer of the headset;
at least one heart rate sensing assembly configured to detect heart rate data of the wearer; and
a wireless communication unit configured to communicate heart rate data to a gateway device.

9. The headset of claim 8, wherein the wireless communication unit is configured to communicate via at least one wireless protocol.

10. The headset of claim 8, where the heart rate sensing assembly comprises a photoplethysmograph sensing assembly.

11. The headset of claim 8, further comprising at least one earpiece configured to be worn on an ear, the heart rate sensing assembly comprising at least one sensor on the earpiece.

12. The headset of claim 11, wherein the at least one sensor is positioned on the earpiece so as to detect heart rate data from at least one of a portion of the ear other than the earlobe, an ear canal, and a region of the wearer other than the ear.

13. The headset of claim 8, further comprising at least one inertial motion sensing device capable of detecting motion of the wearer, the detected motion being usable to reduce motion artifacts attributable to movement of the wearer from the detected heart rate data.

14. The headset of claim 8, further comprising a processing unit configured to receive the detected heart rate data and determine a heart rate of wearer based on the heart rate data.

15. A mobile device for monitoring heart rate of a user, the mobile device comprising:
a handset configured to wirelessly communicate with a headset configured to be worn by the user; and
a processing unit configured to receive heart rate data from the headset and determine a heart rate of user based on the heart rate data.

16. The mobile device of claim 15, further comprising a wireless headset, the wireless headset including at least one heart rate sensing assembly configured to detect the heart rate data.

17. The mobile device of claim 16, wherein the headset includes an audio communication unit configured to output audio hearable by the user.

18. The mobile device of claim 16, wherein the wireless headset further comprises at least one inertial motion sensing device capable of detecting motion of the wearer, the processing unit being configured to reduce motion artifacts attributable to movement of the wearer from the detected heart rate data.

19. The mobile device of claim 15, wherein the handset comprises one of a cellular telephone, a smartphone, a personal digital assistant, personal information device, a pager, an mp3 player, and an iPod, and wherein the handset communicates with the headset via at least one wireless protocol.

20. The mobile device of claim 15, further comprising an auxiliary device configured to wirelessly communicate with at least one of the handset and the headset, the auxiliary device being configured to display information associated with the heart rate data.
